(19) United States
(12) Patent Application Publication
(10) Pub. No.: US 2010/0094132 A1
(43) Pub. Date: Apr. 15, 2010

(54) Ultrasound system having a simplified user interface

(75) Inventors: Joshua M. Hansen, Everett, WA (US); Bradley J. Sliger, Seattle, WA (US); Scott Lockhart, Redmond, WA (US); Thomas Iverson, Kirkland, WA (US)

Correspondence Address: SonoSite, Inc. / Fulbright & Jaworski, L.L.P., 2200 Ross Avenue, Suite 2800, Dallas, TX, 75201, US

(73) Assignee: Sonosite, Inc., Bothell, WA (US)

(21) Appl. No.: 12/249,529

(22) Filed: Oct. 10, 2008

(51) Int. Cl. A61B 8/14

(52) U.S. Cl. 600/443

(57) ABSTRACT

The present invention is directed to a system and method which allows for a simplified user interface tailored to specific procedures and which allows for mobility and easy sanitation. Advantage is taken of an understanding as to the critical functions that an operator is to perform for a specific range of procedures and tailoring the user input to accommodate only those functions. In one embodiment, the user interface consists of a touch screen and a rotatable knob having positional indents with the indents providing incremental increases or decreases in functionality specific to exams or procedures performed with the sonographic equipment. Using this approach, the complexity normally associated with operating sonographic devices is greatly reduced. The operational controls available from the touch screen are changeable to accommodate different function uses of the sonographic device.

### TECHNICAL FIELD

This disclosure relates to ultrasound systems and more specifically to such systems having a simplified user interface where the controls are integral to the display screen.

### BACKGROUND OF THE INVENTION

Medical equipment has become highly complex and with this complexity there is a necessity for training users in the proper operation of the equipment. As complexity increases so does the number of optional settings and adjustments that a user must make in order to have the equipment function properly.

Ultrasound imaging is useful for a wide variety of medical procedures, some of which require complex imaging and thus a sonographer must be highly trained in order to establish and maintain the proper settings throughout the procedure. However, ultrasound also has many uses where it is not practical, or economical, to use highly skilled sonographers. Thus, it is important to design sonography devices that are user friendly even to those who are not highly trained.

Because many of the procedures that “easy to use” sonography devices are called upon to perform are those procedures that are repeated often, sometimes in adverse conditions, such as on a battlefield, in a crowed emergency room of a hospital, or on a medical transportation vehicle, the ability to maintain sanitary conditions in and around the sonographic equipment is of utmost importance.

One procedure for which easy-to-use sonography is used is the Peripherally Inserted central Catheter (“PICC”) procedure. In many situations, a nurse, as opposed to a physician, does the PICC procedure with the aid of images provided by the sonographic equipment. In such procedures, the nurse must maintain a sterile field and has minimal contact with the sonographic equipment during the procedure. This contact is primarily to adjust the depth and gain of the images. Another procedure is similar to use as a visual stethoscope in which sonography is used to find fluid within a patient. This procedure is called a Focused Assessment with Sonography in Trauma (“FAST”) exam in which the operator looks at four quadrants in the body for fluid collection. Again this requires very minimal interaction, and is usually limited to depth and gain.

Another requirement for sonographic equipment designed to perform these functions is that it be easily portable so that it can be used in field situations. It is desirable that military personnel, for example, carry the entire sonographic device in his/her backpack for easy use when scanning injured people in the field.

In some cases the test is as simple as making certain that a fetus is okay after a trauma. The device will be carried in an ambulance and may be hand-carried to the location of an accident victim for quickly observing certain internal functions.

Currently available sonographic equipment is intimidating to use, having a myriad of controls and dials that must be properly set. Those new to sonography with minimal training can easily be intimidated when confronted with a complex user interface.

### BRIEF SUMMARY OF THE INVENTION

The present invention is directed to a system and method which allows for a simplified user interface tailored to specific procedures and which allows for mobility and easy sanitation. Advantage is taken of an understanding as to the critical functions that an operator is to perform for a specific range of procedures and tailoring the user input to accommodate only those functions. In one embodiment, the user interface consists of a touch screen and a rotatable knob having positional indents with the indents providing incremental increases or decreases in functionality specific to exams or procedures performed with the sonographic equipment. Using this approach, the complexity normally associated with operating sonographic devices is greatly reduced. The operational controls available from the touch screen are changeable to accommodate different function uses of the sonographic device.

In one embodiment, there is an obvious power switch that is intuitive to use and non-threatening since it appears to be exactly as one would expect for commonly used electronic equipment, such as a cell phone. Using the intuitively obvious power button along with clear and simple touchscreen controls allows the user to get started in a comfortable manner thereby reducing the intimidation factor that can be caused by operating what, in actual reality, is a fairly complex piece of medical equipment.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

For a more complete understanding of the present invention, reference is now made to the following descriptions taken in conjunction with the accompanying drawing, in which:

FIG. 1 is an isometric front view of one embodiment of a simplified sonographic system;

FIG. 2 is an isometric top view of the simplified sonographic system shown in FIG. 1;

FIG. 3 is a back view of the simplified sonographic system shown in FIG. 1;

FIGS. 4A, 4B and 4C illustrate one embodiment of a port replicator for use with the device shown in FIG. 1; and

FIG. 5 shows an embodiment of a display used in FIG. 1.

### DETAILED DESCRIPTION OF THE INVENTION

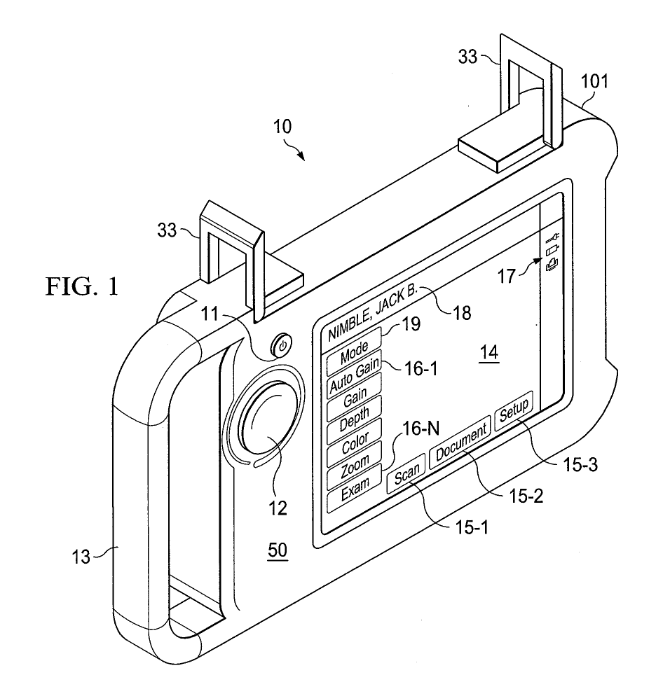

FIG. 1 is an isometric front view of one embodiment of a simplified sonographic system 10. Switch 11 is a power on/off switch and is designed to be similar to on/off switches used on common consumer electronics, such as cell phones and the like. Rotational dial 12 has indents positioned around its periphery. The dial 12 can be a mechanically rotatable switch or it can be a touch sensitive plate response to a human finger moving circularly within its confines. Each different control position operates to adjust the parameters of different features. Dial 12 also can be pressed to actuate a switch and, if desired, to change a parameter. Face 50 is touch sensitive and allows the system to form control areas that can be touched by the user to control various functions. Surrounding the device is protective bumper 101 which can be made from a soft elastomer and which serves to reduce shock resulting from the unit being dropped or placed heavily on a table or stand. The bumper can also act as a hand grip to allow the user to easily carry and position the device. If desired, hinged D-rings 33 can be positioned on one or more edges so that the device can be hung, for example, from a carbineer in a field hospital. In one embodiment, the form factor of device 10 is 11 inches by 8 inches by 2 inches and thus can easily be hand-held.

Bumper 101 can be designed to have different colors, or color patterns and thus can be used to distinguish a device arranged for one operational configuration from a different configuration, or to distinguish a device used by one user from a device used by another user. Bumper 101 can act as a seal around the parameter of the device and help to keep the device sealed to prevent germs and other undesirable material from accumulating in and around the surface of the device.

Note that in the embodiment shown, all controls are located on touchscreen 14 and there are no mechanical controls. The face of the touchscreen is an elastomer bonded to the LCD display. The position of each control is not critical and the feature zones are shown for reference only and can be changed as desired. The type and location of each specific control may, if desired, be customized on a user by user basis. For example, a military physician may require more features than a PICC nurse, emergency service technician (ambulance) or a veterinarian. Note that the physical structure of the housing can be optimized around the function(s) to be performed by the ultrasound device and/or the user. One method for distinguishing a device optimized for a particular user from a device optimized for another user is to change the color of the bumper guard that defines the form factor of the device. Another method for distinguishing device types is to change the form-factor slightly, such as by adding D-rings, a handle, such as handle 13, etc.

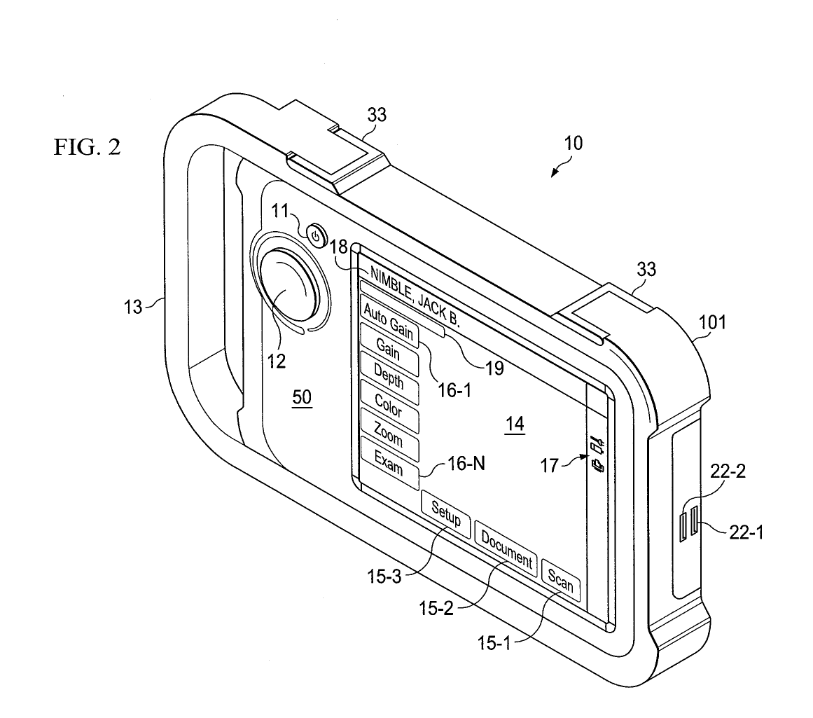

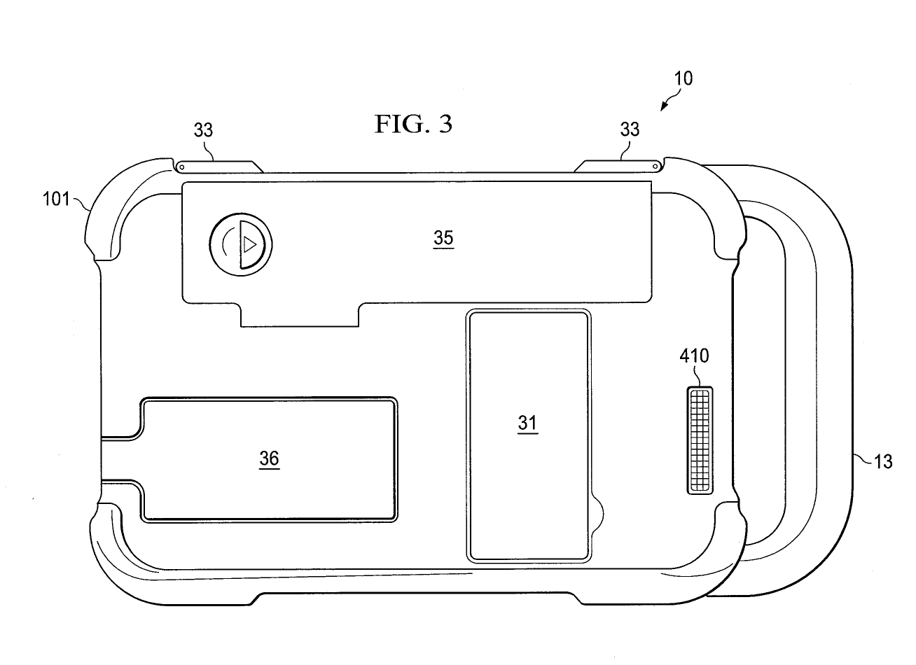

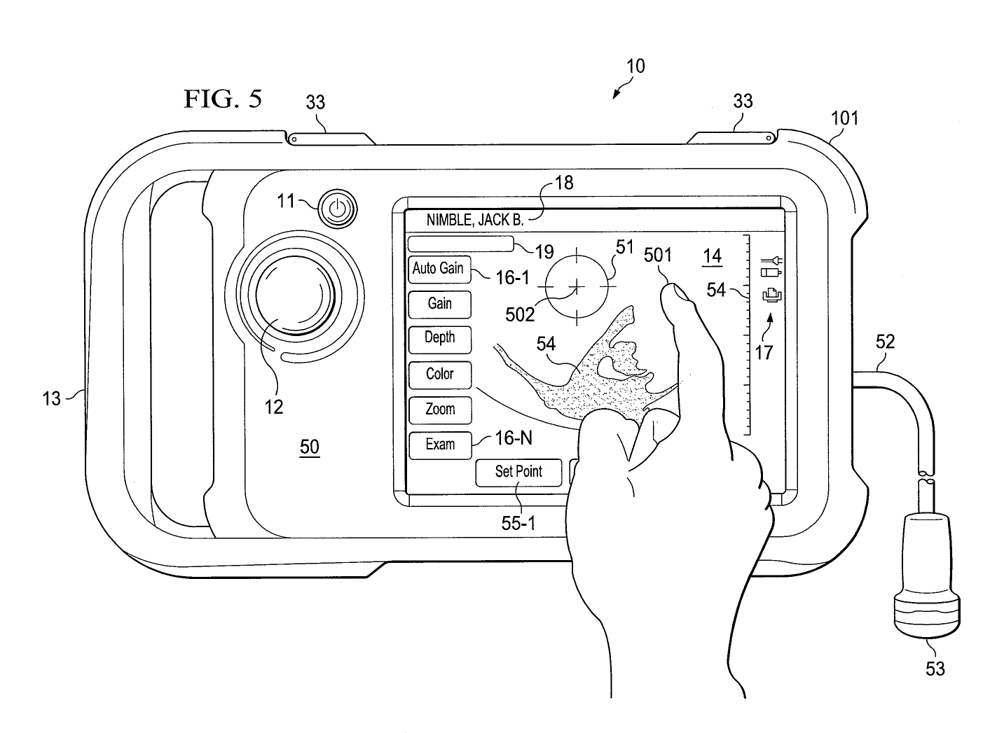

FIG. 2 is an isometric top view of device 10 showing USB ports 22-1 and 22-2. Other interface connectors can be ported through port replicator connector 401 (shown on FIG. 3), and all transducers can be connected to the system via connector 36 (FIG. 3) on the back of the system. Different transducers, such as transducer 53 (FIG. 5), can be used for different procedures, such as cardiac, abdominal, breast, etc. Material 101, facilitates vacuum closure of the device. Device 10 has a battery case 35 which allows for battery placement and storage. The design of device 10 is such that it can be used across many configurations.

FIG. 3 is a back view of device 10 showing kickstand 31 (shown in the folded-in position) which allows device 10 to be positioned on a horizontal surface and allows the viewing angle of face 50 (shown on FIG. 1) to be adjusted as desired. As discussed, handle 13, if added, serves to make device 10 easier to transport by hand. As shown, there are two D-rings 33 that can be folded out (as shown in FIG. 1) to be used in combination with a carabineer or a rope or a clip so that the system can be hung from a line or from a bed, or from a gurney, in a field hospital or the like.

The external configuration of device 10 can be modified (typically in the factory) by removing or adding the bumper, adding or removing the handle, or adding the D-rings, changing bumper color (or partial color). This then allows the factory to pre-build devices and customize the exterior just before shipping. The ability to easily and quickly customize the exterior of the device to a customer's specification results in manufacturing efficiencies thereby reducing the per unit manufacturing costs for sonography equipment and thus increasing the availability of such devices for medical diagnosis.

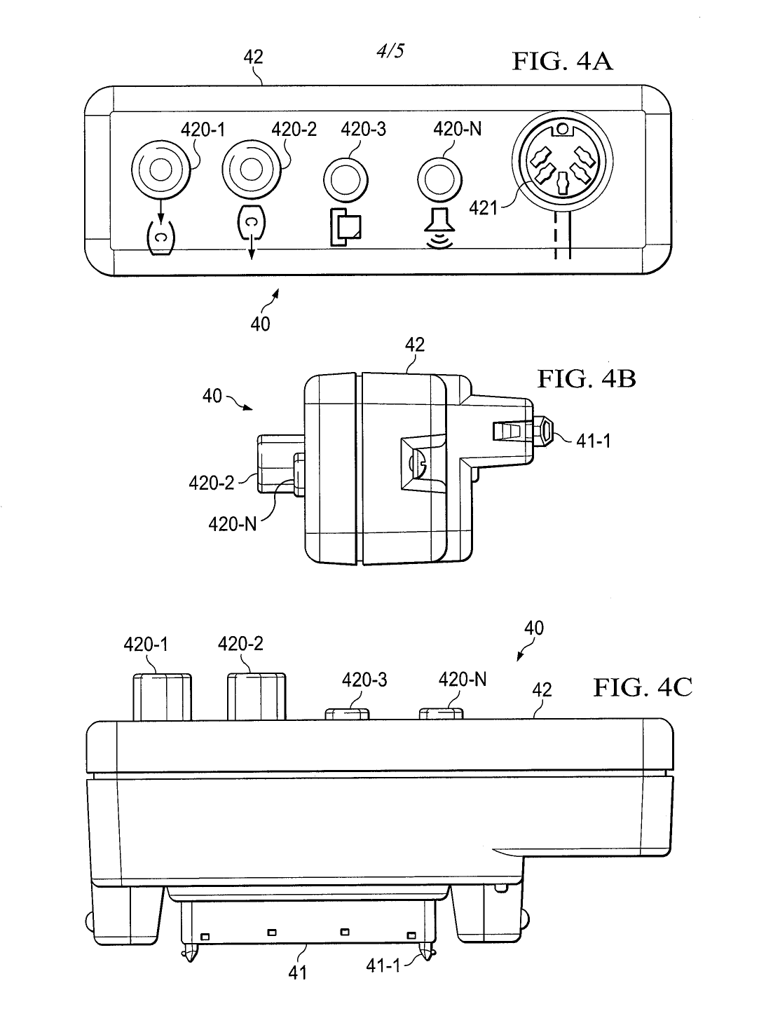

FIGS. 4A, 4B and 4C illustrate one embodiment (top, side and front, respectively) of port replicator 40 for use with the device shown in FIG. 1. The port replicator allows any number of external devices and power to become electronically connected to device 10. Power and recoding equipment are but two of such external devices that can be connected via the port replicator.

The user can plug any number of different peripheral devices, such as a DVR or a printer, or external power, etc) into port replicator 40 using one or more of sockets 420-1 to 420-N or 421. Note that different arrangements of sockets can be installed on different replicators as desired. Any type of socket type can be used as desired. The port replicator allows the peripheral devices to use their normal plugs, such as RCA jacks, miniature or regular audio jacks, or even specialized plugs. Socket 421 is the socket connecting the power cord to the system. The sockets of the replicator are connected to terminals in connector 41 (FIG. 4C) which, in turn, mate with port replicator connector 410 (shown in FIG. 3), on the ultrasound device.

Replicator 40 can be easily released from device 10. Thus, in an emergency situation the operator need not unplug each piece of peripheral equipment from the sonography device but may simply remove the port replicator from device 10 and transport the device to a patient located away from the peripheral devices where device 10 can be operated without connection to the peripheral devices. Once removed from the port replicator, device 10 will run from its own internal power source. In the embodiment shown, peripheral devices, except devices using the USB ports, can only be plugged into the port replicator.

If desired, different port replicators can be used for different operations. For example, one port replicator can be used for testing and/or changing the internal operations of a connected device 10 and another used for patient examination. If desired, different replicators can be tailored to different operating environments, such as a hospital setting or a military field setting.

Returning to FIG. 1, it can be seen that display 14 of device 10 is divided into different sections. Along the right side there is status display area 17 which provides system status information. These display areas indicate to the user various pieces of information, such as what transducer is attached and what examination (procedure) is currently being undertaken based on various factors, such as the active transducer. One area can show battery status and whether or not the system is plugged into a power source. Print and wireless indicators can be employed if desired. System banner 18 is shown along the top of the display, and displays the patient's name, and any other information desired such as patient ID number, institution name, physician initials, date and time.

System mode data is displayed in the upper left hand corner of the display, in box 19. This data includes system mode (such as 2D), current optimization setting, image enhancement settings, color mode (if enabled) and a color bar (if color is enabled).

The high level system controls, such as controls that the user will interact with in preparation for or during a procedure are located, in this embodiment, on the bottom of the screen. This includes controls (such as 15-1 to 15-N) to initiate scanning, view patient information, view system setup and, if desired, to freeze (not shown) a current scanned image.

Secondary system controls 16-1 to 16-N are located along the left side of the display. Some of these controls are menus that allow the user to select various operations such as depth, gain and auto-gain. The auto-gain uses, in one embodiment, an algorithm to look at all the parameters that are set. Based on the totality of the settings the system will provide a “best guess” for the settings for the gain settings for a particular image. The auto-gain parameters can be over-ridden by the operator if desired. Two of the controls, namely the depth and gain controls, are selectively connected to rotary dial 12. In this manner, when either depth or gain is selected by the user, movement of the rotary dial will increase or decrease the selected parameter. The use of the rotary dial serves several purposes. First, it allows fine motor control for fine adjustment. Second, it is easy to grasp by feel alone so that the user need not move his/her eyes from the display in order to find a “soft” button. Third, rotary control provides tactile and sensatory feedback as to relative movement without the need for lines and scales. The indents of dial 12 allow the user to feel (and perhaps hear) the relative movement of the rotary dial thereby increasing the usefulness and ease of use of the dial.

Central area 14 of display 50 is where the image, as obtained via the ultrasound transducer (not shown) is displayed. The user may change the displayed image by manipulating the depth and/or gain buttons in conjunction with the rotary dial. Along the side of display 14 can be, if desired, a depth scale calibrated, typically, in centimeters.

In operation, if the user desires to see images already collected the user will select the freeze button or the patient button and use secondary menu controls, located along the left side of the touchscreen to view the images. In the multi-image display mode, the rotary knob would then be used to scroll though images. If desired, the rotary knob could have menu items in a circle around the knob. Each menu item would then select what metric the knob will control. The user would turn the knob to select the desired function and then, for example, push in the knob for actual selection of the function. The display presentation would follow the selected menu item. This then avoids having the user touch the screen for selection because touching the screen leaves a residue which degrades sonographic imaging thereby reducing image quality.

When it is desired to present color, such as with blood flow, a box comes onto the screen and the user moves the box to the area where the color is desired. In one embodiment, the user can simply drag the box to the desired location by a finger touch or by using a stylus. The stylus (or a finger) can also be used to measure an object simply by touching the object at the end points of the object to be measured. The portion between the touched end points can then be dragged to the measuring scale, such as to scale 54 (FIG. 5). If desired, to avoid physically touching the screen a stylus could be used to define the area to be moved and/or measured. The measurements can, for example, be displayed along the bottom of the touchscreen, just above the primary controls.

Another feature of the system is that the user can touch the screen to enable a zoom effect (gain) for a portion or the entire image. Also, the movement of the finger (stylus) could cause a change in depth under the skin line to change, perhaps with the depth being dependant upon the movement of the finger along the screen or upon pressure or proportional to the number of quick screen taps by the user.

FIG. 5 shows an aspect of system 10 in which a cursor pointer is offset from a finger. The curser is positioned over the point of interest by moving the finger. In this manner, the finger does not obscure the point of interest. In the embodiment shown, finger tip 501 allows a user to select a point of interest, such as point 502 and cross-hair 51 (or any other pointer mechanism) appears on the screen at that point. Cross-hair 51 is offset from the user's finger so as not to obscure the point of interest.

In operation, the user would select the Modes >Caliper button (not shown) and cursor pointer 51 would appear on the screen. The user can point anywhere on the screen, move his/her finger to move the cursor. When the first point is found, the user presses ‘set point 1’ (55-1). The user then moves the pointer by moving his/her finger to the second position on the image and presses ‘set point 2’ (not shown). A dotted line would then appear between the points and a measurement readout will appear on the screen.

Also shown in FIG. 5 is sonographic transducer 53 connected to the system by cable 52. Any number of different transducers can be used with different cable types if desired. The system can have software code stored on storage media as desired or the system can be controlled by ASICs or a combination of ASICs and software. The software would be run on a processor (not shown) and could be changed, if desired, from time to time.

The system may be mounted to a stand which will allow the user to position the system at different heights and angles. It will also allow the user to store transducers, gel, keyboard and other supplies in a basket or on a tray. The stand will also provide storage locations for a printer, DVD/DVO storage device, bar code reader or other peripheral equipment. The stand will allow the system to be easily moved to different locations. It will also have a quick release mechanism so that the user can quickly remove the system from the stand in order to carry it to another location. The system is mounted to the stand, or other mounting devices such as wall mount articulated arms, using the standard VESA mounting configuration.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

What is claimed is:

1. A hand-held sonographic device comprising:
a frame having a tablet form-factor, said frame being surrounded by an energy absorbing bumper;
a front panel positioned within said frame; and
a set of controls integral with said front panel touchscreen display, said controls adapted for allowing a user to control a specific set of tasks to obtain sonographic images, said task set being less than the full complement of tasks available to be performed by said sonographic device, said integral controls maintaining a opening-free front panel.

2. The hand-held device of claim 1 wherein said bumper is a soft elastomer.

3. The hand-held device of claim 1 wherein said front panel is attached to said frame.

4. The hand-held device of claim 3 wherein said form-factor is approximately 11 inches by 8″ by 2 inches.

5. The hand-held device of claim 2 wherein one of said controls is a clearly defined on-off switch.

6. The hand-held device of claim 5 further comprising
a rotary control positioned within said frame, said rotary control controlling different functions at different times, said function being controlled at any point in time being dependant upon the operation of at least one of said integral controls during that period of time.

7. The hand-held device of claim 6 wherein rotation of said rotary control serves to provide fine tuning to said function being controlled.

8. The hand-held device of claim 5 further comprising
a rotary control positioned within said frame, said rotary control controlling different functions at different times,
said rotary control having various indents selectable by rotation of said rotary control and wherein said function being controlled at any point in time being dependant upon the prior selection of one of said indents.

9. The hand-held device of claim 8 wherein rotation of said rotary control serves to provide fine tuning to said function being controlled.

10. The hand-held device of claim 1 further comprising:
a handle connected to said frame, said handle extending said form factor of said frame when connected to said frame.

11. The hand-held device of claim 1 further comprising:
means for allowing a user to change a color of said bumper from time to time.

12. The hand-held device of claim 1 further comprising:
means for allowing said bumper to act as a sanitary seal around a periphery of said frame.

13. A method for using an ultrasound device, said method comprising:
selecting by a user an ultrasound device pre-tailored for a specific function;
selecting by said user one of a few controllable functions appropriate for said specific function; and
fine tuning said selected function by rotating a dial common to a plurality of selectable functions.

14. The method set forth in claim 13 wherein said controllable functions are selected from the list of:
depth, gain, auto-gain, freeze-frame, save.

15. The method of claim 13 wherein said ultrasound device is portable with a notebook form-factor common to a plurality of different specific functions and wherein said selecting comprises:
observing by said user a color of a bumper guard defining at least a portion of said form-factor.

16. The method of claim 15 further comprising:
removing a specific colored bumper guard by said user.

17. The method of claim 13 wherein said ultrasound device is portable with a notebook form-factor common to a plurality of different specific functions and wherein said selecting comprises:
adjusting said form factor by said user to accommodate handling of said ultrasound device.

18. The method of claim 17 wherein said adjusting comprises at least one of the following actions:
adding D-rings; adding a kickstand support, adding a handle.

19. The method of claim 13 further comprising:
measuring a sonogram image using a scale located on a side of a display for measurement purposes, said display positioned within said form factor.

20. An ultrasound device, said device comprising:
means for allowing a user to select an ultrasound device pre-tailored for a specific function;
means for allowing a user to select one of a few controllable functions appropriate for said specific function; and
means for allowing said user to fine tune said selected function by rotating a dial common to a plurality of selectable functions.

21. The device of claim 20 wherein said controllable function select means is contained in a easily sanitizable continuous surface structure.

22. The device of claim 20 wherein said controllable functions are selected from the list of:
depth, gain, auto-gain, freeze-frame, save.

23. The device of claim 20 further comprising:
a portable housing having a notebook form-factor common to a plurality of different specific functions and wherein said selecting means comprises:
a bumper guard having a particular color, said bumper guard defining at least a portion of said form-factor.

24. The device of claim 23 further comprising:
means for allowing a user to select said colored bumper guard.

25. The device of claim 20 further comprising:
a portable housing having a notebook form-factor common to a plurality of different specific functions and wherein said selecting comprises:
means for allowing said user to adjust said form factor to accommodate handling of said ultrasound device.

26. The device of claim 20 further comprising:
a display positioned within said form factor; and
means for allowing a user to measure a sonogram image using a scale located on a side of said display for measurement purposes.

27. The device of claim 20 further comprising:
a port replicator, said port replicator operable for allowing power to be supplied to said device when said port replicator device is positioned within said device; and
means for allowing a user to remove said port replicator from said device such that said device, at least temporarily, operates on a stand-alone basis from power supplied from within said device.
